(19) United States
(12) Patent Application Publication
(10) Pub. No.: US 2011/0301673 A1
(43) Pub. Date: Dec. 8, 2011

(54) LIGHT THERAPY SYSTEMS AND METHODS

(75) Inventors: Kevin W. Hoffer, Portland, OR (US); Michelle L. Kvernmo, Tigard, OR (US); Elizabeth Langvin, Sherwood, OR (US); Andrew A. Owings, Portland, OR (US); Bradley W. Wilkins, Tigard, OR (US)

(73) Assignee: NIKE, Inc., Beaverton, OR (US)

(21) Appl. No.: 13/151,587

(22) Filed: Jun. 2, 2011

Related U.S. Application Data
(60) Provisional application No. 61/350,844, filed on Jun. 2, 2010.

(51) Int. Cl. A61N 5/06

(52) U.S. Cl. 607/91

(57) ABSTRACT

Light therapy systems and methods improve the use of healthy athletes' muscles before, during, and while recovering after athletic activity. LEDs increase blood flow, increase cell metabolism, and increase waste removal from treated areas of the user's body. Systems and methods for providing these benefits to the user are disclosed herein.

### REFERENCE TO RELATED APPLICATION

This application claims priority to co-pending U.S. Provisional Patent Application No. 61/350,844 filed on Jun. 2, 2010, entitled, “Light Therapy Systems and Methods,” which is entirely incorporated by reference herein.

### FIELD

The present disclosure relates to light therapy systems and methods incorporated into an article of manufacture and/or an article of apparel. More specifically, articles of manufacture and apparel include light-emitting diodes that provide physiological benefits to an athlete before, during, and while recovering after athletic activity.

### BACKGROUND & SUMMARY

Light therapy provides significant physiological benefits to humans and has been used by medical professionals to treat various conditions and injuries such as soft tissue injuries, edema, wound healing, diabetes, nerve damage, cancer, joint pain, jaundice, skin treatments, etc. Typically, light therapy is administered to patients at a medical facility or for home use under the supervision of medical professionals after an accident or injury or during treatment of a condition.

Various types of light sources have beneficial results in light therapy including lasers and light-emitting diodes (LEDs). In particular, LEDs have proven beneficial as a light source for light therapy because of their ability to emit various wavelengths of light that offer many physiological benefits to humans and their relative low cost to own, maintain, and replace.

To realize the benefits of LED therapy, some systems and methods incorporate LEDs into an article of manufacture or an article of apparel. As discussed above, these articles of manufacture and apparel are designed for use in a medical treatment facility or home use with a medical treatment purpose and are not designed for a healthy or active user with comfort and versatility in mind. The fabrics and other materials that are used in these articles often are bulky and are designed for use when the patient is sedentary or otherwise relatively stationary. Further, these LED therapy systems require extensive physical wiring for connecting the LEDs to a power source. Known LED therapy articles are not able to detect the wavelength of the light being emitted from the LEDs nor do they include a timing device to prevent overexposure of the patient to LED therapy.

Healthy athletes may benefit greatly from the disclosed LED therapy systems and methods. During all activities in which athletes engage, and especially during strenuous activities, the performance of athletes' muscles decreases over time due to a decrease in the presence of oxygen, a decrease in cell metabolism, a decrease in the ability to remove waste from the area, and other reasons. LED therapy for athlete's muscles anytime before, during, and/or after athletic activity may increase blood flow to the treated area (which increases oxygen delivery to that area), may stimulate cell metabolism, and may increase waste removal. Each of these benefits may result in overall improvements to the performance of athletes' muscles and a faster recovery during and after muscle use. Additionally, LED therapy provides warmth to the athlete's muscles. Known ultrasound techniques also provide such warming effects of the athlete's muscles, but fail to provide any of the additional physiological benefits of LED therapy that are described above.

The present invention overcomes these and other shortfalls with the disclosed LED therapy systems and methods. In an example embodiment, an article of manufacture comprises a first layer of material, a second layer of material and at least two LEDs. The first layer defines at least a first cavity and a second cavity. Both the length and width of the first layer exceed the height of the first layer. A first LED is positioned in the first cavity and a second LED is positioned in the second cavity. The first LED and the second LED are electronically coupled to a power source. The second layer is secured to the first layer such that the second layer extends over at least the first cavity and the second cavity.

In another embodiment, an article of manufacture comprises a first layer of material, a second layer of material, and a plurality of LEDs. The first layer defines at least two columns located parallel to each other and a plurality of cavities located along each of the two columns. Each of the LEDs is positioned within one of the plurality of cavities. The second layer is secured to the first layer such that the second layer extends over each of the plurality of cavities.

This section provides a general summary of the disclosure, and is not a comprehensive disclosure of its full scope or all of its features. Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 illustrates a top view of an exemplary embodiment of the disclosed LED therapy systems and methods.

FIG. 2 illustrates an exemplary embodiment of an LED therapy system embodied in a leg wrap.

FIG. 3 illustrates a cross-section of a cavity in which an LED is positioned, according to some exemplary embodiments.

### DETAILED DESCRIPTION

An article of manufacture comprises: (1) a first layer of material defining at least a first cavity and a second cavity, wherein both the length and width of the first layer exceeds the height of the first layer; (2) at least a first LED positioned in the first cavity and a second LED positioned in the second cavity; and (3) a second layer of material secured to the first layer such that the second layer extends over at least the first cavity and the second cavity. In this article of manufacture, the first LED and the second LED are electronically coupled to a power source.

The article of manufacture is any suitable article. For example, the article of manufacture may be a wrap, an article of apparel, a blanket or other covering, a medical device, or the like. In another example, the article of manufacture is a wrap for a user's leg, specifically the user's lower leg. In yet another example, the article of manufacture is athletic apparel. The article of manufacture optionally includes a securing mechanism that is selectively securable. The securing mechanism permits the user to easily detachably secure the article of manufacture to the user's body by opening or “making larger” a particular section of the article. For example, a wrap having two, opposing ends includes a securing mechanism that secures the two ends together when the wrap is applied to (wrapped around) a portion of the user's body and allows the two ends to separate so the user can easily remove the wrap from the user's body. In another example, a pair of pants has a securing mechanism at the waist to permit the user to easily put on and remove the pants by increasing the size of the opening of the waist for the user. The securing mechanism includes any one or more of the following, including, but not limited to, hook and loop fasteners, buttons, snaps, buckles, magnets, adhesives, strap(s) that tie or otherwise can be secured together, or the like.

The first layer of the article of manufacture is any suitable material, including, but not limited to any synthetic material. The material of the first layer has any suitable characteristics. In some examples, the material of the first layer is breathable and stretchable. In other examples, the material of the first layer is incorporated into an article of manufacture that is attached to a person and thus includes any suitable characteristics that make the material comfortable and easily wearable for the person. Other example characteristics include, but are not limited to, hypo-allergenic materials, fluid-wicking materials, materials having a comfortable feel against a user's skin, flexible and resilient materials, etc.

The first layer of material in the disclosed article of manufacture has a first side and an opposing, second side. The first layer of material defines at least a first cavity and a second cavity. The first cavity and the second cavity may define six sides. In an example embodiment, each side of a six-sided cavity is positioned at 90° with respect to one another. In another example, the first cavity and the second cavity have rounded or curved edges. In still other examples, the first cavity has sides that are positioned 90° with respect to each other and the second cavity has sides with rounded or curved edges. The construction of the cavities may all be uniform or may differ in any suitable fashion. One of each of the sides of the first cavity and the second cavity is open and faces toward the first side of the first layer of material. The open side of the first cavity and the second cavity face toward the same side of the first layer of material.

The first cavity and the second cavity may be positioned in any suitable configuration on the first layer of material. In one example, the first cavity and the second cavity are positioned adjacent each other. In another example, the first cavity and the second cavity are each positioned along opposing edges of the first layer of material. In yet another example, the first cavity and the second cavity are positioned in a pattern.

Some examples include an article of manufacture in which the first layer defines a first column in which the first cavity and the second cavity are positioned on opposite sides of the first column. In this example configuration, the first cavity is either aligned with the second cavity across the width of the first column or the first cavity is offset from the second cavity across the width of the first column, similar in configuration to cavities (either aligned or offset) in an ice-cube tray. The first layer of material optionally defines any number of columns. Any suitable number of cavities may be positioned along any of the one or more columns.

For example, the article of manufacture has two columns, each having a first side and a second, opposing side. A first portion of a plurality of cavities is located along the first side of the first column and a second portion of the plurality of cavities is located along the second side of the first column. The columns are a straight line, a curved line, or any suitable configuration. In the examples with multiple columns, the columns are positioned parallel, perpendicular, or any other angle with respect to each other. In the example having multiple columns, each of the columns may be a similar configuration or one or more of the columns may have a different configuration than another one or more of the columns.

The height of any of the cavities is equal to or less than the height or thickness of the first layer of material. In some examples, the height of the cavities defines (is equal to) the height of the first layer of material, which reduces the amount of excess material and thus the bulk of the article of manufacture. As described below, the first layer has a first side and a second side. The cavities are entirely enclosed on the second side and have at least a partial opening on the first side to permit positioning of the LED(s) into the cavities. In these examples, the cavities do not define through holes, although this may be incorporated into other example constructions.

In some examples, the first layer is generally flat having a height that is equal to or slightly greater than the height of the tallest cavity. The first layer has a length and width that exceeds its height. The first layer is generally a thin sheet of material that is shaped to suit the article of manufacture. For example, in the leg wrap configuration, the length and width of the first layer define the surface area of the wrap itself such that it is a suitable size, shape, and contour for wrapping around the wearer's leg.

As described above, the article of manufacture also comprises at least a first LED and a second LED. The first LED is positioned in the first cavity and the second LED is positioned in the second cavity. The article of manufacture includes any number of LEDs. In an example configuration, the number of LEDs is equal to or less than the number of cavities defined in the first layer of material. In other examples, more than one LED are positioned within any one or more cavities defined in the first layer of material. The size and shape of each of the LEDs are uniform or may vary. In an example, the LEDs are sized and shaped to fit snugly within the cavities. For example, the cavities are a three-dimensional cube shape and the LEDs are square and flat. The shape of these LEDs complements the shape of the cavities.

The LEDs are capable of emitting light at any desirable wavelength. In some examples, the light emitted from the LEDs is within the range of 600-1200 nanometers (nm). In more specific examples, the wavelength of the light emitted from the LEDs is within the range of 800-1000 nm. Even more specifically, the wavelength of light emitted from the LEDs is 935 nm. Generally, LEDs emit light with a spectrum of wavelengths. The spectrum may have a “peak” wavelength and one or more other wavelengths having a smaller wavelength than the “peak” wavelength. For example, one or more of the LEDs emit a light having a “peak” wavelength of 935 nm and a plurality of wavelengths between 800-935 nm. The spectrum may include a narrow or a broad “peak.” A narrow peak would include a single or a small range of wavelengths. A broad peak would include a larger range of wavelengths. The physiological benefits vary depending on the value(s) of the wavelength(s) within the “peak” and the type of wavelength (i.e., narrow v. broad) within the “peak.”

The first LED may emit light at a first wavelength and the second LED may emit light at a second wavelength within the ranges described above. In one example, the first LED and the second LED emit light having a peak wavelength of 935 nm. The wavelength of the light emitted from the LED is dependent upon the type of semiconductor materials that are used to manufacture the LEDs. In turn, the wavelength corresponds to the “color” of the light. In this example, wavelengths of light between 600-1200 nm correspond approximately to orange, red, and infrared light. Light having wavelengths of 800+ nm is near-infrared or infrared light. Different wavelengths of light cause different physiological benefits to the users. The LEDs emit any wavelength of light that results in a physiological benefit to the user.

In some examples, an article of manufacture includes a first portion of a plurality of LEDs that emit light that includes a first wavelength and a second portion of the plurality of LEDs that emit light that includes a second wavelength. The article of manufacture includes a controller that permits the user (or any other switching operation) to adjust the wavelength of light therapy being emitted from the article of manufacture by switching power to one or the other of the first portion or second portion of LEDs. For example, the first portion of LEDs emits light at a wavelength of 800 nm and the second portion of LEDs emits light at a wavelength of 935 nm. A switch controls power to both the first portion and the second portion of LEDs and gives the user the option to adjust the wavelength being emitted from the article of manufacture by switching power on to the LEDs emitting the wavelength of choice (e.g., 935 nm) and switching power off to the LEDs emitting the other wavelength (e.g., 800 nm). Any number of portions of LEDs can be included in the article of manufacture. For example, the article of manufacture has four portions of the plurality of LEDs, each portion of LEDs emitting a different wavelength of light than the other three portions, and a controller that allows a user to select the wavelength of light that he or she wants to use in the LED therapy. Any suitable means to control the LEDs that receive power can be used.

The article of manufacture optionally includes a sensor(s) that measures the wavelength of the light that is emitted from any one or more of the LEDs and/or a sensor(s) that measures the level of power being output from the power source. The sensor(s) also may compile the data relating to the measurement of the wavelength of light that is emitted by one or more of the LEDs and/or the data relating to the level of power being output from the power source. This information may trigger a signal to the user (or any other person) that the light being emitted from one or more of the LEDs is not within the range that results in the most beneficial physiological results for the user and/or that the power source is not outputting the power necessary to operate the LEDs at the desired wavelength(s). In one example, once the sensor measures light emitted from an LED that is outside of the predetermined range of wavelengths (e.g., any of the ranges described above that provide the user with physiological benefits from the light therapy) or that the power source is not emitting the requisite amount of power, the signal indicates to the user that: (1) the LED(s) needs to be replaced; and/or (2) the power source needs to be checked to confirm that the necessary amount of power is being generated to power the LEDs to emit light within the predetermined range of wavelengths. In another example, the measurements obtained from the sensor are compiled and stored in a memory that is also operably secured to the article of manufacture and are used for any suitable means. The sensor is any suitable sensor that is capable of measuring light emitted from a light source and/or any suitable sensor that is capable of measuring power output from a power source. More than one sensor is optionally included in the article of manufacture.

The article of manufacture optionally further comprises a timer that controls the length of time that the first LED and/or the second LED emit light. The timer causes the LEDs to start and stop emitting light. The timer can serve as both a time-monitoring device and/or an on-off switch for the LEDs. For example, the timer causes the power source to stop powering the LEDs after a predetermined amount of time. More specifically, the predetermined amount of time of exposure to the LEDs for the user is ten minutes in some examples. The timer tracks the amount of time that lapsed since the LEDs began emitting light. In this example, after ten minutes the timer causes the LEDs to stop emitting light. The timer may be set to cause the LEDs to stop emitting light at any predetermined time interval. In some examples, the timer is coupled to the sensor and causes the LEDs to stop emitting light if the wavelength of the light is measured to be outside of the predetermined range of wavelengths that result in physiological benefits to the user and/or if the power source outputs power below a predetermined threshold level.

As described above, the article of manufacture also comprises a second layer of material that is secured to the first layer such that the second layer extends over at least the first cavity and the second cavity. The second layer is secured to the first side of the first layer of material and extends over the openings of the cavities to enclose or “house” the LEDs within the cavities. The second layer includes any suitable material such as thermoplastic polyurethane (TPU). The second layer is optionally transparent so that the light emitted from the LEDs is applied as directly as possible to the user's body. The height of the second layer is small and relatively thin and may (1) permit as much of the light emitted from the LEDs as possible to contact the user's body; (2) provide moisture management qualities to wick moisture away from the user's body; (3) provide heat evaporation qualities to help the user's body expend heat away from the surface of the user's body, and the like. The material(s) selected for the second layer may include any suitable properties.

In an example, the second layer is one or more separate and discrete elements covering the openings of the cavities. Alternatively, the second layer is a single sheet of material that extends over the entire surface area or any portion thereof of the first side of the first layer of material. In this example, the second layer has a surface area that is approximately equal to (or at least extends over a majority of) the surface area of the first layer. The second layer is secured to the first layer in any suitable fashion, such as via adhesive, bonding, cementing, gluing, and/or any suitable mechanical fastener. In some examples, the second layer is selectively releasable from the first layer to provide easy access to the LEDs positioned within the cavities when they need to be replaced or maintained.

The first LED is housed within a space defined by the first cavity and a first portion of the second layer that extends over the first cavity. The second LED is housed within a space defined by the second cavity and a second portion of the second layer that extends over the second cavity. The spaces within which the LEDs are housed are enclosed by the second layer, which is operably secured to the first layer with any suitable adhesive, bonding agent, or the like. The adhesive, bonding agent or the like helps protect the LEDs by preventing moisture, debris, and any other undesirable elements from entering the spaces in which the LEDs are housed.

In the disclosed articles of manufacture, the first LED and the second LED are electronically coupled to a power source. In some examples, the first and the second LED are electronically coupled to the power source in series and in other examples, they are electronically coupled to the power source in parallel. The power source is any suitable power source. For example, the power source is a battery that is operably secured to the article of manufacture. This example configuration would permit the article of manufacture to be portable, which gives the wearer the freedom to move around while using the article of manufacture. The article of manufacture includes any suitable battery that is able to provide the LEDs with sufficient power to emit light within the predetermined range of wavelengths (e.g., between 600-1200 nm). In an example, the article of manufacture includes a battery that generates an alert when its power output begins to decline in a manner that would decrease the level of light emitted from the LEDs such that its wavelength falls outside of the predetermined range of wavelengths.

In another example embodiment, an article of manufacture comprises a first layer of material defining at least two columns located parallel to each other and a plurality of cavities; a plurality of LEDs that each are positioned within one of the plurality of cavities; and a second layer of material secured to the first layer such that the second layer extends over each of the plurality of cavities. A first portion (one or more) of the plurality of cavities is located along the first column and a second portion (one or more) of the plurality of cavities is located along the second column. In some examples, cavities are located on opposite sides of a column, either directly opposite each other or offset from each other.

The LEDs and the power source are electronically coupled to each other in any suitable fashion. In an example, a lightweight, electrically conductive material is etched onto the first layer such that the LEDs are connected to the conductive material when the LEDs are placed in their respective cavities. In another example, the electrical connections coupling the LEDs to each other and the power source can include one or more semiconductor wafers containing microelectronics and optionally electronics that control power to and from one or more of the LEDs. These example embodiments reduce the weight and increase the flexibility of the article of manufacture.

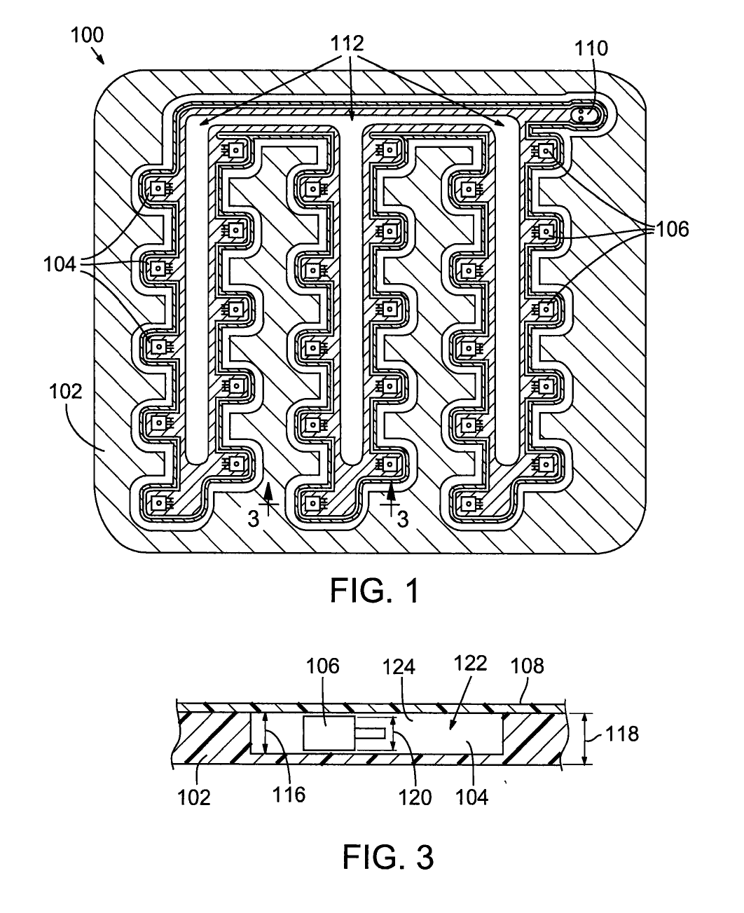

Specific systems and methods can be implemented that incorporate one or more of the embodiments described above. Referring now to the specific examples illustrated in FIGS. 1-3, a leg wrap 100 includes a first layer 102 of material that defines a plurality of cavities 104. The first layer 102 is relatively thin and has a surface area that is approximately rectangular in shape. The leg wrap 100 also includes a plurality of square, flat LEDs 106 that are each positioned within respective cavities 104 of the first layer 102. Each of the LEDs 106 emits light that includes a “peak” wavelength of 935 nanometers. A second layer 108 of material is secured to the first layer 102. The second layer 108 is approximately the same size and shape as the first layer 102. In this example, the second layer 108 is transparent and is thinner (has a smaller height) than the first layer 102. The LEDs 106 are electronically coupled to a portable power source 110.

As illustrated in FIG. 1, the first layer 102 includes three, parallel columns 112 that are equally spaced apart. Five cavities 104 are positioned along each side of each of the three columns 112. Cavities 104 along opposite sides of each of the three columns 112 are offset from each other. Each of the plurality of LEDs 106 is electronically coupled to each other in a series configuration. A portable power source 110, such as a battery or the like, completes the circuit such that the power source 110 is positioned in a continuous loop with each of the LEDs 106. In this example, the first layer 102 is etched with electrically conductive materials such that the LEDs 106 are electronically coupled to each other and with the power source 110. In this example, the first layer 102 includes a material that is breathable and stretchable such that it is able to be efficiently wrapped around a wearer's leg 114. The second material 108 includes a TPU.

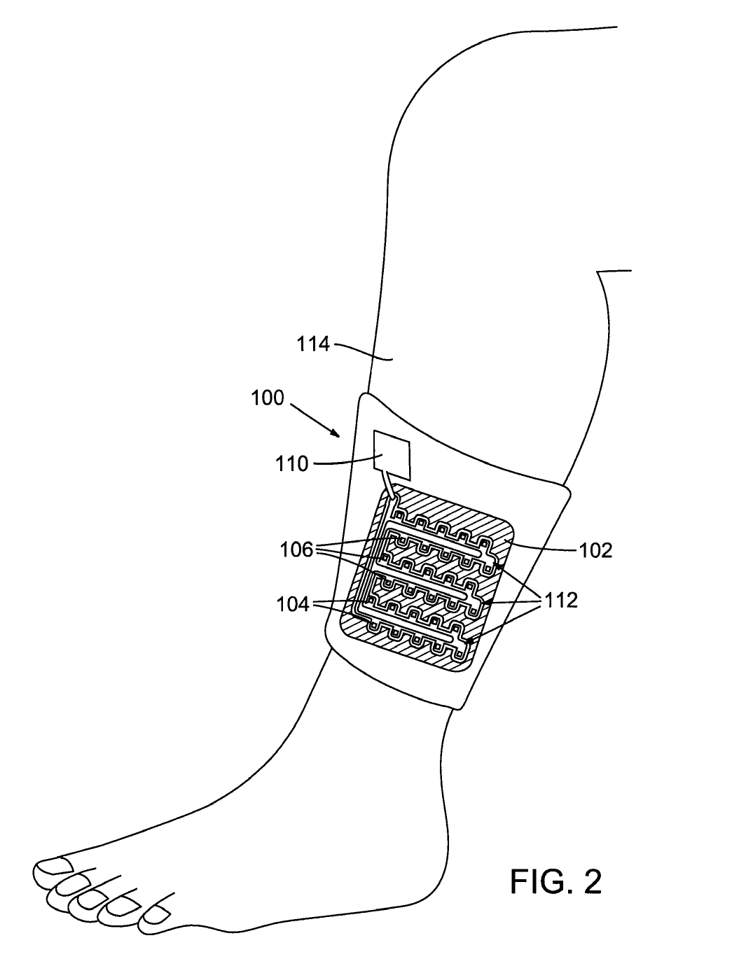

FIG. 2 illustrates the leg wrap 100 worn by a wearer on the wearer's lower leg 114. This exemplary embodiment of the leg wrap 100 is relatively snugly fit to the wearer's leg 114. In the example embodiment illustrated in FIG. 2, the LEDs 106 in the leg wrap 100 are positioned such that they emit light toward the lateral portion of the wearer's lower leg 114. In other examples, the LEDs 106 are positioned to emit light toward any desired portion of the wearer's leg 114, including entirely surrounding any portion(s) of the wearer's leg 114.

FIG. 3 illustrates a cross-sectional view of a cavity 104 including the first layer 102, an LED 106 positioned within the cavity 104, and the second layer 108 extending over the cavity 104. In this example, the height 116 of the cavity 104 is less than the thickness 118 of the first layer 102, i.e., the cavity 104 does not extend through the entire thickness 118 of the first layer 102. The height 116 of the cavity 104 exceeds the height 120 of the LED 106. The cavity 104 defines a space 122 that has a greater volume than the volume of the LED 106 in this example. In other examples, the cavity 104 defines a space 122 that is only slightly larger than the LED 106 such that the LED 106 fits snugly within the cavity 104. The cavity 104 defines an opening 124 that is entirely covered by the second layer 108 such that the cavity 104 and the second layer 108 completely enclose the space 122 within the cavity 104.

An athlete uses one of the systems described above to apply LED light therapy to a portion of his or her body before, during, and/or after activity. In an example, the athlete uses this system in preparing for an activity, such as warming up for practice or competition in a sport. This is useful when the athlete is performing this activity when the air temperature is cold or when the athlete's muscles need to be warmed up efficiently and/or quickly in preparation for the activity to achieve peak performance. When the LED system is applied to the athlete prior to the athlete engaging in the activity, the athlete may realize multiple benefits including, but not limited to: warm muscles; increased blood flow to the treated area resulting in an increase in oxygen delivery to the same area; an increase in cell metabolism and removal of waste from this process; and the like.

In another example, an athlete competing in a basketball competition rests during halftime of the game. During halftime, the athlete wears pants that incorporate one or more embodiments of the LED system described above to help the athlete's leg muscles recover from the strain, damage, and/or fatigue they endured during the first half of the game. The pants are tightly fitted to the athlete's body so that the maximum amount of LED light can reach the user's skin during the therapy. Any alternative embodiment of the pants also can be used, such as looser fitting pants. The LED system may help warm the athlete's muscles, enhance blood flow to the area being treated, improve cell metabolism, and improve the rate of waste removal. Enhanced blood flow to the treated area of the athlete's body results in greater delivery of oxygen to that area. These benefits may provide athletes with a recovery tool that improves performance of the athlete after treatment. In this case, the athlete's muscles are healthier after the LED therapy during the second half of the basketball game, which enhances the athlete's performance.

In a disclosed method of enhancing performance of an athlete, the athlete engages in a first portion of a first activity. Then the athlete rests. During the resting time for the athlete, an LED light therapy system is applied to at least a portion of the athlete's body. The athlete then engages in a second portion of the activity. The athlete may choose to remove the LED light therapy system before beginning the second portion of the activity or may keep the LED light therapy system on his or her body during a portion or all of the second activity. In this example, the LED light therapy system is a pair of pants that apply the LED light therapy to at least a portion of the athlete's legs or a wrap that can be secured around at least a portion of the athlete's legs. The exemplary LED light therapy system used in this method is portable, i.e., it has a battery that is operably secured to the system. The portable nature of this exemplary LED light therapy system allows healthy athletes to use the LED light therapy system before, during, and after activities to realize the full physiological benefits to their muscles.

In another example, an athlete wears an article of athletic apparel that has a recess sized to receive any embodiment of the article of manufacture described above. The recess can be a pouch or pocket and can be either secured or detachably secured to the article of athletic apparel. For example, the recess is a pocket that is sewn into a portion of the pant leg of an athlete's warm-up pants. When the athlete is resting during a break in athletic activity (e.g., halftime, a timeout, being taken out of the game, etc.), the athlete wears the pants and easily inserts a wrap or other embodiment of the above described article of manufacture into the pocket. In an alternative example, the pocket is detachably secured to the warm-up pants with one or more hook and loop fasteners or any other suitable mechanical connector. The alternative example has the option to include a power source that is operably secured to the article of manufacture or that is attachable to the article of manufacture. Any suitable power source is suitable for this embodiment.

In another alternative example, the article of manufacture is made integral with the article of athletic apparel, such as by sewing the article of manufacture into a portion of a leg of warm-up pants or basketball shorts. These exemplary embodiments are light-weight and easily flexible so that the athlete can perform the activities without restriction from the article of manufacture.

The foregoing description of the embodiments has been provided for purposes of illustration and description. It is not intended to be exhaustive or to limit the invention. For example, although the disclosed embodiments are leg wraps and articles of apparel, those skilled in the art will understand that the disclosed light therapy systems and methods may be embodied in any suitable article of manufacture. For example, the disclosed systems and methods may be included in a wrap for the arms, torso, head, or any other portion of a user's body; a hat or other head covering; and any other article of manufacture that is capable of delivering the light therapy to a user.

Similarly, individual elements or features of a particular embodiment are generally not limited to that particular embodiment, but, where applicable, are interchangeable and can be used in a selected embodiment, even if not specifically shown or described. The same also may be varied in many ways. Such variations are not to be regarded as a departure from the invention, and all such modifications are intended to be included within the scope of the invention.

We claim:

1. An article of manufacture, comprising:
a first layer of material defining at least a first cavity and a second cavity, wherein both the length and width of the first layer exceed the height of the first layer;
at least a first light-emitting diode capable of emitting a first light having at least a first wavelength, the first light-emitting diode positioned in the first cavity and a second light-emitting diode capable of emitting a second light having at least a second wavelength, the second light-emitting diode positioned in the second cavity; and
a second layer of material secured to the first layer such that the second layer extends over at least the first cavity and the second cavity,
wherein the first light-emitting diode and the second light-emitting diode are electronically coupled to a power source.

2. The article of manufacture of claim 1, wherein the article of manufacture is a leg wrap.

3. The article of manufacture of claim 1, wherein the first layer of material is breathable and stretchable.

4. The article of manufacture of claim 1, wherein the second layer of material includes thermoplastic polyurethane.

5. The article of manufacture of claim 1, wherein the first wavelength and the second wavelength are different.

6. The article of manufacture of claim 1, wherein the first wavelength and the second wavelength are substantially the same.

7. The article of manufacture of claim 1, wherein the first wavelength and the second wavelength are within a range between 800 and 1000 nanometers.

8. The article of manufacture of claim 1, wherein the first wavelength and the second wavelength are approximately 935 nanometers.

9. The article of manufacture of claim 1, wherein the first layer defines a first column having a first side and an opposing, second side, and wherein the first cavity is positioned on the first side and the second cavity is positioned on the second side of the first column.

10. A method of enhancing performance of an athlete, comprising the steps of:
instructing an athlete to detachably secure a light therapy device to the athlete;
instructing the athlete to operate the light therapy device during a first portion of an athletic activity; and
instructing the athlete to cause the light therapy device to cease operating during a second portion of the athletic activity.

11. The method of enhancing performance of an athlete of claim 10, where the first portion of athletic activity is a rest period for the athlete.

12. The method of enhancing performance of an athlete of claim 10, where the first portion of athletic activity requires a first level of exertion to the athlete and the second portion of athletic activity includes a second level of exertion to the athlete that is different than the first level of exertion.

13. The method of enhancing performance of an athlete of claim 10, where the first portion of athletic activity is a rest period and the athlete is instructed to detachably secure the light therapy device to the athlete at the beginning of the rest period.

14. The method of enhancing performance of an athlete of claim 10, further comprising the step of instructing the athlete to remove the light therapy device.

15. A method of maintaining peak performance of an athlete, comprising the steps of:
detachably securing a light therapy device to the athlete;
operating the light therapy device during a defined break in an athletic activity; and
causing the light therapy device to cease operating prior to the athlete returning to the athletic activity following the defined break.

16. The method of maintaining peak performance of an athlete during a defined break in an athletic activity of claim 15, where the light therapy device is the article of manufacture as recited in claim 1.

17. The method of maintaining peak performance of an athlete during a defined break in an athletic activity of claim 15, wherein the light therapy device is a leg wrap detachably secured to the athlete's leg.

18. The method of maintaining peak performance of an athlete during a defined break in an athletic activity of claim 15, where the light therapy device is a light-weight, self-contained, portable structure.

19. The method of maintaining peak performance of an athlete during a defined break in an athletic activity of claim 15, where the light therapy device is stretchable.

20. The method of maintaining peak performance of an athlete during a defined break in an athletic activity of claim 15, where the athletic activity is a sports event between at least two teams, and the athlete is a player on one of the teams.

21. The method of maintaining peak performance of an athlete during a defined break in an athletic activity of claim 15, where the step of detachably securing a light therapy device to the athlete is performed before the athletic activity begins.

22. The method of maintaining peak performance of an athlete during a defined break in an athletic activity of claim 15, where the step of detachably securing a light therapy device to the athlete is performed at the beginning of the defined break.

23. The method of maintaining peak performance of an athlete during a defined break in an athletic activity of claim 15, further comprising the step of removing the light therapy device from the athlete prior to the athlete returning to the athletic activity following the defined break.
